(19) United States
(12) Patent Application Publication
(10) Pub. No.: US 2006/0136031 A1
(43) Pub. Date: Jun. 22, 2006

(54) Balloon deployable stent and method of using the same

(76) Inventors: Richard Gallo, Montreal (CA); Patrick Terriault, Verdun (CA); Vladimir Brailovski, Montreal (CA)

Correspondence Address: Louis Tessier, P O Box 54029, Town of Mount-Royal, QC, H3P 3H4, CA

(21) Appl. No.: 10/533,118

(22) PCT Filed: Oct. 29, 2003
(86) PCT No.: PCT/CA03/01676

§ 371 Date: Nov. 9, 2005

Related U.S. Application Data
(60) Provisional application No. 60/422,489, filed on Oct. 31, 2002.

(51) Int. Cl. A61F 2/84; A61F 2/90

(52) U.S. Cl. 623/1.11; 623/1.15; 623/1.19

(57) ABSTRACT

The present invention provides a balloon-deployable stent having a progressive expansion over time and a method for using such a stent, thereby reducing restenosis. The stent has a progressive radial expansion of an armature (12) comprising a material having an elasticity allowing the self-deployment of the armature and of a matrix (14) comprising a second material having a rigidity and a conformation allowing a retention of the armature in a contracted position. The stent is deployed with the help of a balloon delivered into the armature, which allows an irreversible deformation of the matrix during the inflation of the balloon and enables a radial expansion of the armature.

### FIELD OF THE INVENTION

The present invention generally relates to stents. More specifically, the present invention relates to a balloon deployable stent and to a method making use thereof.

### BACKGROUND OF THE INVENTION

Stents are typically used to enlarge or to liberate a passageway in a vessel or a lumen.

For example, cardiovascular stents are used to increase a diameter of a partially obstructed cardiovascular artery by forcing an enlargement thereof through deployment of a metallic structure. FIG. 1 illustrates the functioning mode of a cardiovascular stent, as is well known in the art.

The installation of a cardiovascular stent is a well-established technique in the art. More than 500,000 angioplasties per year are performed worldwide.

Two main materials are available in the marketplace for the manufacture of cardiovascular stents: stainless steel and nitinol.

In the case of stainless steel stents, an inflatable balloon causes the deformation of the stent. The stent, in its contracted state, is mounted on the balloon and introduced into the human body. When the contracted stent mounted on the balloon is positioned at a target location, the balloon is inflated, which results in a plastic deformation of the stent. Next, the balloon is deflated and pulled out of the artery, leaving the stent in a deployed configuration against the walls of the artery. FIG. 2 illustrates such a stainless steel stent deployed and contracted on an inflatable balloon.

Nitinol stents take advantage of an intrinsic property of shape-memory alloy, whereby this material always regains an original shape thereof if bent. The nitinol stent is introduced into a catheter, which keeps the stent in a contracted position, and moved in an artery to a target location. Once in position, the stent is mechanically expulsed from the catheter and thereby enabled to take its predetermined completely deployed configuration, without the need for an inflatable balloon. Nitinol stents are therefore self-deployable. FIG. 3 shows a nitinol stent, which adopts its completely deployed form upon expulsion from the catheter.

According to the “Handbook of Coronary Stents” (edited by P. W. Serruys, Martin Dunitz Ltd, London, 1997), nitinol has been employed since 1997 in a number of stents. In its superelastic regime, nitinol is able to accommodate deformations in the order of 8% and tends to completely regain its initial non-deformed state. In comparison, stainless steels such as the alloy 316L, which is frequently used to manufacture stents, are able to accommodate a reversible elastic deformation of about 0.1%. The elastic domain of nitinol is approximately 80 times larger than that of conventional metals like steel and aluminum. FIG. 5 schematizes the superelastic behavior of nitinol, where E is the elasticity module; ε_{mf} the strain at the end of the transformation; σₘₛ the stress at the beginning of the transformation; σ_{af} the constraint at the end of the transformation; and H the transformation module. Besides such a level of reversible elastic deformations, nitinol has a high resistance comparable to that of a metallic material, which allows an adequate dilation of the artery and also guaranties stability over time.

A main concern is related to the fact that current installation procedures of either the stainless steel or the nitinol stents still cause a certain trauma of the vascular walls.

For example, the pressure exerted by the inflatable balloon for the installation of the stainless steel stents, so that the latter espouses the inner walls of the vessel, traumatizes the artery. One of the main problems associated with the use of stainless steel stents as illustrated in FIG. 3 is that an over-expansion is necessary during the inflation of the balloon to compensate for an elastic springback effect. Indeed, when the balloon is deflated, the stainless steel has a tendency to contract, or to springback, due to an elastic component of the total deformation. Consequently, to position the stent against the wall of the artery in the best possible way, generally the stent needs be inflated up to a diameter superior to that of the vessel in order to compensate for the shrinkage or springback during deflation. Such an over-expansion may damage the artery even further and contribute to restenosis, while sub-expansion of a stainless steel stent diminishes the interference constraint between the stent and the walls, and may be detrimental since it may be accompanied with increased rates of thrombosis and vessel occlusion and, consequently, provoke the loss of stent-artery contact. This effect is still more prominent if the artery relaxes with time or if its diameter augments because of different physiological reasons.

As far as nitinol stents are concerned, their diameter, once completely deployed, may be greater than that of the artery. Hence, during deployment, the nitinol stent is in contact with the artery and the equilibrium of forces between the latter and the stent is attained for a smaller diameter, thereby creating a permanent but light pressure on the walls of the vessels. Indeed, due to the particular behaviour of the nitinol stent, the stent keeps applying a light pressure, which is practically constant while the diameter of the artery increases, and continues to do so until the stent attains its completely deployed diameter. Alternatively, should the diameter of the artery decrease because of a spasm for example, the stent offers a significant resistance to such a contraction. However, the instantaneous liberation of a self-deploying nitinol stent may also provoke an impact on the inner walls of a vessel and, hence, causes trauma.

Traumas due to installation of the stents may contribute to restenosis phenomenon (e.g. recurrence of vessel narrowing at the site previously dilated). According to studies, about 30% of angioplasties present a degree of restenosis within the first 6 months. As a result, a second intervention, for example an introduction of another stent or a major brachytherapy operation with the goal of effectuating bypass surgery, is needed. Restenosis generates important costs for the healthcare system. It would therefore be advantageous to provide a cardiovascular stent that minimizes the trauma imposed on the coronary or peripheral vascular systems during the deployment thereof.

It would therefore be advantageous to provide a balloon deployable stent that does not necessitate an over-expansion during placement overcomes the drawbacks associated with sub-expansion.

Table 1 presents advantages and limitations associated with stainless steel stents, such as illustrated in FIG. 2, and nitinol stents, such as illustrated in FIGS. 3 and 4.

TABLE I

| Stainless Steel Stents | | Nitinol Stents | |
|---|---|---|---|
| (FIG. 2) | | (FIGS. 3, 4) | |
| Advantages | Limitations | Advantages | Limitations |
| Possibility of | Necessary use | Self-deploying | Instantaneous |
| gradually | of inflatable | behaviour which | deployment often |
| applying | balloon | avoids the use of | affects its |
| pressure on | Over-inflation | the inflatable | positioning and |
| the walls of the | may be | balloon | may traumatize |
| arteries | necessary to | Applying | inner walls of |
| because of the | compensate for | permanent | the vessel |
| inflatable | elastic | pressure on walls, | Elastic behavior |
| balloon | springback | hence, no elastic | after its |
| Malleability | Loss of | springback | installation, |
| after | pressure on the | | which limits |
| installation, | inner walls of the | | interventions on |
| which is | artery because of | | the secondary |
| beneficial for | the elastic | | branches |
| operations on | springback | | |
| the secondary | | | |
| branches | | | |

In summary, stainless steel stents satisfy the gradual deployment property. Their installation method with an inflatable balloon allows a precise and gradual positioning. However, they generally require an over-deployment and often suffer from elastic springback. Nitinol stents, on the other hand, may adapt to variations of the vessel diameter. Nevertheless, their self-deploying capacity and abrupt deployment may compromise their positioning.

A stainless steel stent requires an inflatable balloon to deform, and once deformed, it does not tend to regain its initial contracted state, whereas a nitinol self-deployable stent always tends to regain its completely deployed state, without the need for an inflatable balloon. Indeed, as shown in FIG. 3, when the nitinol stent is expulsed from the catheter, which keeps it in a contracted position, it deploys itself instantly to come back to its initial state. Lastly, this capacity to accommodate a great deformation facilitates the progression of the stent through the often tortuous vessels (e.g. arteries and other lumens) of the human body.

Therefore, there is still a need for a stent, which gradually deploys, allowing a precise and controlled installation while avoiding an abrupt mechanical action, and which, once deployed, exerts a continuous pressure on the walls of the artery or vessel even if a diameter thereof increases, through a controlled radial expansion, thereby minimizing the downside of an elastic springback effect.

### OBJECT OF THE INVENTION

The present invention therefore relates to an improved balloon deployable stent and to a method making use thereof.

### SUMMARY OF THE PRESENT INVENTION

The present invention provides a balloon-deployable and controlled radially expandable stent comprising an armature comprising a first material having an elasticity allowing an expansion over time of the armature; a matrix comprising a second material having a rigidity and a conformation allowing a retention of said armature in a contracted position; the stent being deployed with the help of a balloon introduced into the armature, the balloon allowing an irreversible deformation of said matrix during inflation of the balloon and allowing expansion of the armature.

The invention further provides a method of angioplasty in an artery of a patient comprising: introducing and positioning in a vessel of the patient a self-deploying stent having a progressive deployment comprising an armature comprising a material having an elasticity allowing self-deployment of the armature; and a matrix comprising a second material having a rigidity and a conformation allowing a retention of the armature in a contracted position; deploying the armature using a balloon delivered in the armature, the balloon ensuring an irreversible deformation of the matrix during inflation of the balloon and allowing a self-deployment of the armature; and removing the balloon from the vessel; whereby a progressive self-deployment of the armature allows a positioning of the armature at a predetermined position and a diminution of a risk of restenosis.

Other objects, advantages and features of the present invention will become more apparent upon reading of the following non-restrictive description of embodiments thereof, given by way of example only with reference to the accompanying drawings.

### BRIEF DESCRIPTION OF THE DRAWINGS

FIG. 1, which is labeled “prior art”, illustrates the way by which a cardiovascular stent according to the art is inserted into a partially blocked artery (A); the deployment of the stent (B); the enlarged artery (C);

FIG. 2, which is labeled “prior art”, shows a stainless steel stent according to the art deployed (upper section) and contracted on an inflatable balloon (bottom section);

FIG. 3, which is labeled “prior art”, illustrates a nitinol stent according to the art covered by a catheter (upper section); starting to self-deploy as the catheter is withdrawn (towards the left, in the middle section); and completely deployed after expulsion of the catheter (bottom section);

FIG. 4, which is labeled “prior art”, illustrates a) a Radius™ stent comprising five zigzag segments; b) a photo of the stent taken with a scanning microscope demonstrating the precision by a laser cut;

FIG. 5, which is labeled “prior art”, illustrates the superelastic behavior of nitinol;

FIG. 6 illustrate embodiments of a polymeric matrix on the metallic armature according to the present invention;

FIG. 7 schematizes a stress-strain relationship employed in the calculations for the design of the polymeric matrix of a stent according to the present invention

FIG. 8 is a graphic representation of the armature's external diameter evolution;

FIG. 9 illustrates an embodiment of a polymeric ring: A) front view; B) lateral view;

FIG. 10 illustrates polymeric rings braided over-under around the armature according to an embodiment of the present invention;

FIG. 11 illustrates polymeric rings secured into slots provided on the armature according to a further embodiment of the present invention;

FIG. 12 illustrates graphically, the functioning of the stent comprising the nitinol armature and of the polymeric rings, in which A corresponds to the completely deployed stent; B corresponds to the contracted armature; C corresponds to the non-deformed rings; D corresponds to the established equilibrium between the armature and the rings (passage from C to D), the armature passing from B to D; the deployment of the balloon brings the metallic armature from point D to point E and the rings from points D to F. The elastic springback of the polymeric rings is illustrated by the passage from point F to point G, while the armature passes from point E to point G; and

FIG. 13 illustrates graphically the variation in rigidity of the ring caused by the creep property; I corresponds to the rotation of the slope in relation to the pivot; H corresponds to the new equilibrium position.

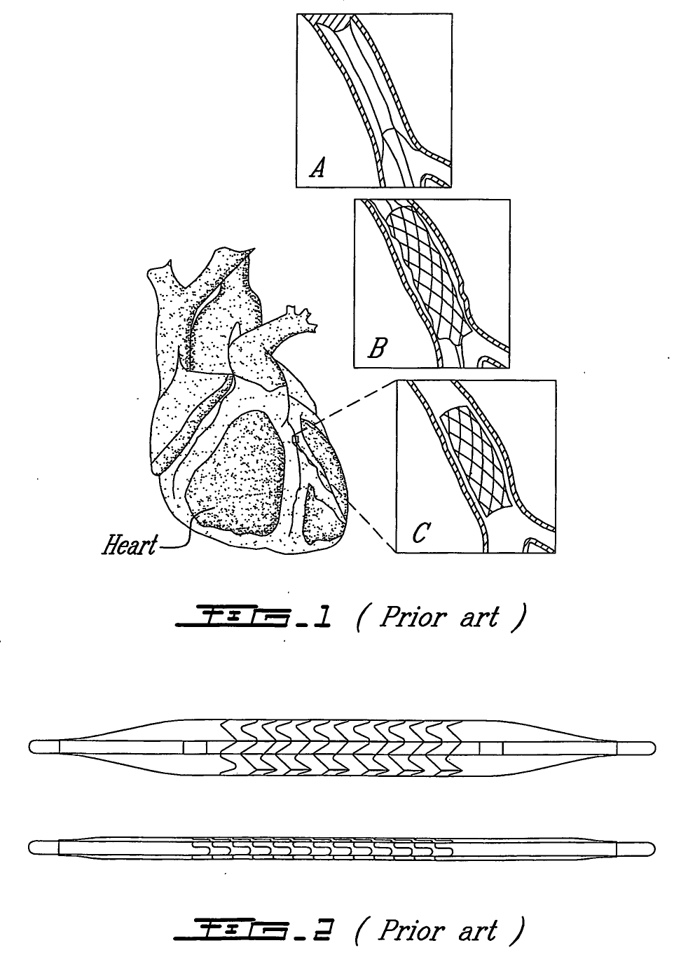

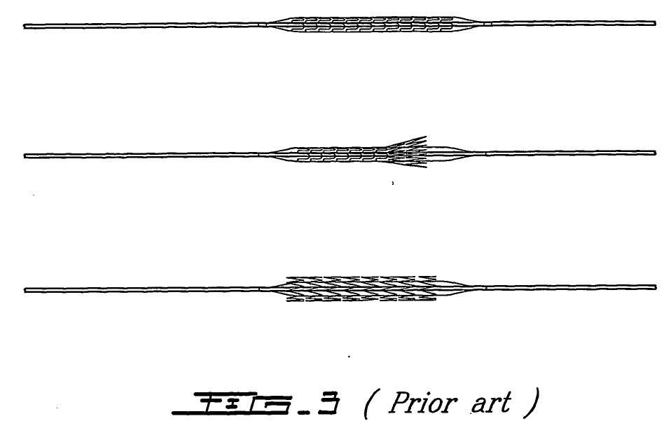

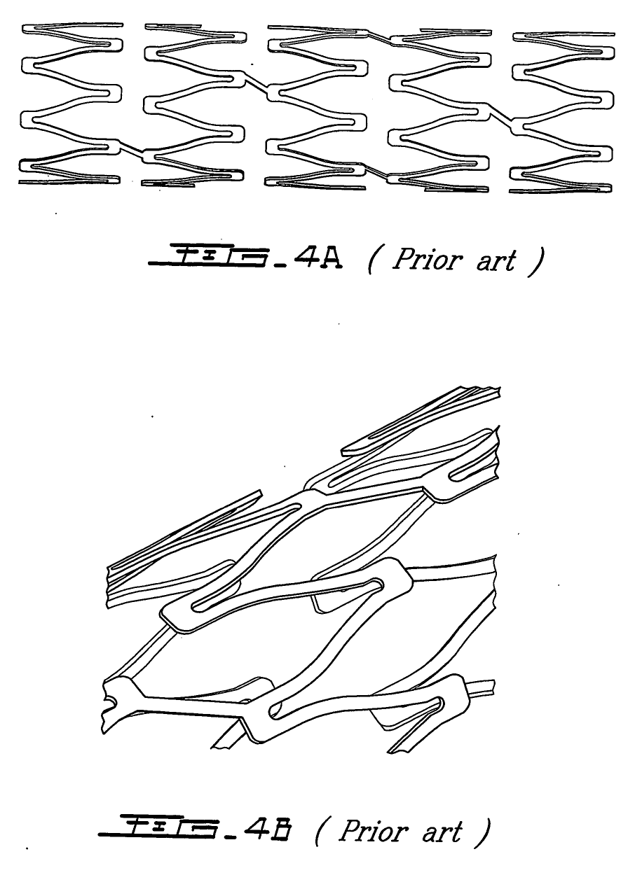

### DETAILED DESCRIPTION OF THE INVENTION

The present invention provides a balloon deployable stent, which has the property of having a progressive radial expansion over time, and a method making use thereof.

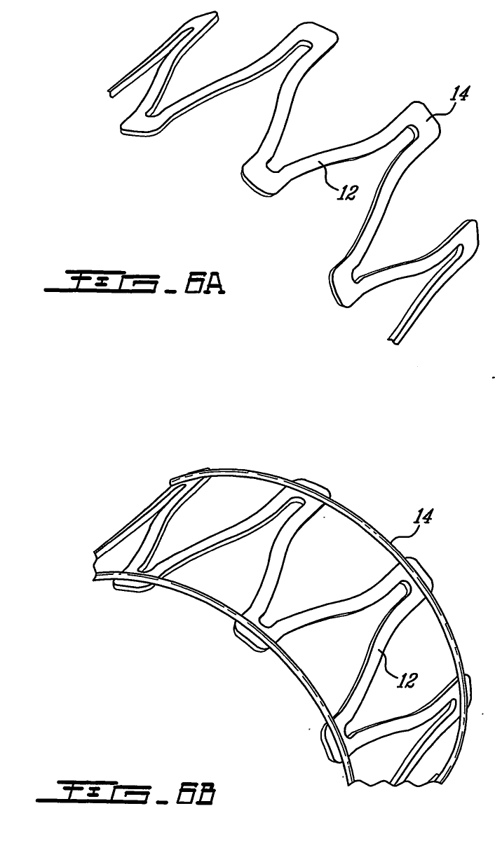

According to a first aspect of the present invention, as illustrated in FIG. 6, a balloon deployable stent according to the present invention comprises an armature 12 and a matrix 14.

The matrix 14 is mounted, either glued or affixed for example, to the armature 12 in a contracted state thereof.

The armature 12 is made of a first material, which may be selected in order to obtain radio-opaque and rigid properties in the artery, which may prove to be interesting properties in the course of an angioplasty intervention, for example, which are comparable to those of metal.

The matrix 14 is made in a second material, which may be selected between materials which, in time, gradually lose their mechanical properties, thereby allowing a gradual and controlled expansion of the armature 12.

The stent of the present invention may further comprise a retention sheath made of a third material. Non-limiting examples of this retention sheath include a sheath per se, as well as a polymer, collagen-like or biological glue material, which inhibits the expansion of the armature 12 and of the matrix 14.

It is to be understood that the term “material” as used herein for the armature 12, matrix 14, sheath or other element of a stent according to the present invention refers to at least one material, and thus covers the combination of many materials, e.g. an alloy, a mix of polymers, a mixture, etc. Obviously, when mixtures of materials are used, the properties of the mixtures satisfy the requirements associated with the particular uses of the stent. For example, when a mixture of materials is used for the matrix 14, at least one of the materials in the mixture loses its mechanical properties in time, thereby enabling a gradual expansion of the armature 12 over time.

The armature 12 may be made in a metal such as nitinol, which is a shape memory alloy comprising titanium and nickel. In this case, the nitinol armature 12 coupled to the matrix 14 is installed on an inflatable balloon to facilitate the installation. The matrix 14, which tends to loose its mechanical properties, allows securing the stent maintained in place on the balloon thereby delaying the deployment thereof. When the balloon is inflated, it deforms the matrix 14 in an irreversible fashion. Having lost its mechanical property, the matrix 14 then no longer restricts the self-deployment of the nitinol armature 12.

It is to be noted that the matrix 14 may be selected in such a way that the loss of its mechanical properties occurs at a relatively low temperature, such as the temperature of the human body (370C).

Furthermore, creep properties of the material(s) of the matrix 14 may be put to use in order to delay the deployment of the stent after the positioning of the stent. It may be briefly reminded that, when a mass is suspended at the end of a wire constituted from a certain material, an instant elongation proportional to the suspended mass may be observed. This elongation is the manifestation of the material's elastic behavior. If this mass is left in place for a sufficient amount of time, a progressive increase in the elongation of the wire over time may be recorded. This progressive elongation is the manifestation of what is referred to as the creep phenomenon. The additional lengthening of the wire is thus an indicator that the material weakens under the creep. For metallic materials, creep generally takes place at high temperatures, (e.g. in the order of several hundreds degrees Celsius). In the present invention, a material exhibiting creep at human body temperature may be selected.

The matrix 14 of the progressive radially expandable stent of the present invention may comprise at least in part polymeric materials, which may creep at low temperatures. In selecting the polymer to make the matrix 14, the following properties are contemplated:

1) a sufficient rigidity allows maintaining the stent in a contracted position on the balloon. During the storage of the stent, a retention sheath (or catheter) may be added, e.g. a hollow cylinder in which for example the stent, including the matrix, mounted on the balloon, may be introduced, so as to avoid an unnecessary creep of the matrix during the storage of the stent. The stent may be maintained in a contracted position when not in the retention sheath, providing the matrix is sufficiently rigid. A polymer with a module of sufficient rigidity may then be selected in order to keep the deployment of the armature;

2) a capacity of plastic deformation of the polymer during dilation caused by the inflation of the balloon, i.e. without elastic return (or possibly with a negligible elastic return), may allow to avoid or minimize a return of the armature towards its contracted position. Therefore, a polymer with a low yield strains (passage from the elastic to the plastic regime), relative to certain silicones from which the reversible elastic strains are several hundred percents, may be advantageous;

3) a capacity of plastic deformation of the polymer during the inflation of the balloon, without tendency of rupturing or fissuring of the polymeric matrix, which might liberate particles in the vessel (e.g. in the blood) may be obtained providing a sufficiently high ultimate strain. It is believed to be within the reach of a person of ordinary skill in the art to select the materials for the armature and matrix so as to be compatible with their clinical use;

4) a capacity to creep at human body temperature under the forces generated by the armature may further contribute to a progressive deployment over time;

5) a biocompatibility and conformance with USP standards is required so that the stent may be used into the human body as an implant. Non-restrictive examples of the different classes of polymer used for medical purposes and which satisfy the USP standards comprise certain types of Urethane-polycarbonate (Bionatel for example), of polycarbonate (Makrolonl for example), of polyethylene or of polypropylene (from Huntsman or Montell for example).

As a way of example, the polymer Makrolonl Rx 2530 (Bayer) has proven to satisfy the previously enunciated features. A high density polyethylene like DMDA-8920 Natural 7 provided by Petromont, and characterized by a rigidity of about 1000 MPA, a yield strain of about 7%, an ultimate strain of over 400% and creep properties whereby about 90% of the initial rigidity is lost after 100 hours at 37° C. under a stress of 8.7 MPA, may also be contemplated for example.

Generally stated, the matrix of the present invention may be made of a polymer having a high rigidity of at least 1000 Mpa, a low yield strain below about 8%, a large ultimate strain over about 100%, and creep properties allowing a minimum loss of 50% of the initial rigidity.

It is to be noted that alternative materials, which loose their mechanical properties over time, may be used to form the matrix, such as materials subject to biodegradation for example.

Therefore, according to a second aspect of the present invention, a method for angioplasty comprises introducing and positioning in a vessel of the patient a self-deploying stent having a progressive deployment comprising an armature comprising of a material having an elasticity allowing self-deployment of said armature and a matrix comprising a second material having a rigidity and a conformation allowing a retention of the armature in a contracted position; deploying the armature using a balloon delivered in the armature, the balloon ensuring an irreversible deformation of the matrix during inflation of the balloon and allowing a self-deployment of the armature; and removing the balloon from the vessel; whereby a progressive self-deployment of the armature allows a positioning of the armature at a predetermined position and a diminution of the risk of restenosis.

Numerical analyses were carried out in order to size the polymeric matrix 14 to be added to the metallic armature 12 and to verify if, theoretically, the armature-polymer unit may pursue a progressive and retarded deployment after having been dilated by the inflatable balloon.

Rather than attempting to size the polymeric matrix 14 by considering simultaneously every desired function, such as initial rigidity, creep property, for example, first calculations were carried out considering only the mechanical behavior of the polymer so as to meet the two following requirements: i) keep the armature 12 in a contracted position before the deployment of the inflatable balloon, and ii) deform itself irreversibly, i.e. by plastic deformations, during the inflation of the balloon without reaching the ultimate strain.

The creep behavior of the polymer will be verified to ensure that the stent offers an additional progressive deployment after installation

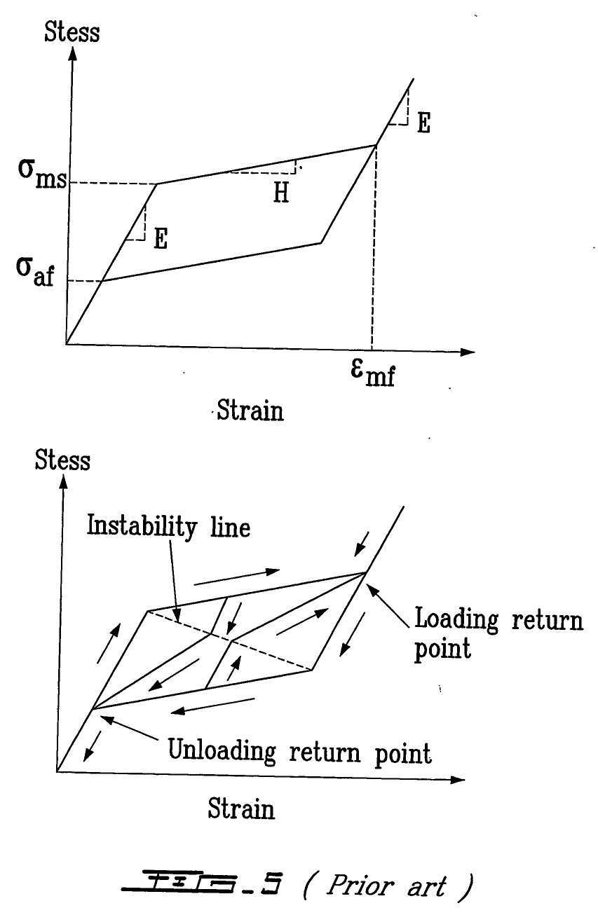

The stent used for the numerical validation is a nitinol a self-deploying Radius™ of SciMED, fabricated from a tube cut to a desired geometry with a laser. This geometry comprises a number of zigzag segments linked to each other by three bridges, as illustrated in FIG. 4, drawn from the Handbook of Coronary Stents, supra.

The dimensions used for the numerical validation are presented in Table 2.

TABLE 2

| | | |
|---|---|---|
| Nominal diameter | 3.0 | mm |
| External diameter completely deployed | 3.75 | mm |
| Width of a zigzag segment | 2.5 | mm |
| Number of zigzags per segment | 9 | |
| Thickness of the stent (tube wall) | 0.11 | mm |
| Width of the strut | 0.11 | mm |

It should be noted that even if the diameter of the completely deployed nitinol armature is 3.75 mm, the company indicates that it is a stent of 3.0 mm. Therefore, it is recommended to use a stent of 3.75 mm for use into an artery of about 3.0 mm.

The properties of nitinol depend greatly on the alloy composition and of the thermal treatment it has received. Average properties as derived from the literature are used for the numerical simulations. The stress-strain relationship, also called the material law, used to stimulate the superelastic behavior of nitinol and the value of the parameters describing this behavior are respectively shown and given in FIG. 5 and Table 3.

TABLE 3

| | | |
|---|---|---|
| Elasticity modulus (E) | 80 000 | MPa |
| Poisson coefficient (υ) | 0.3 | |
| Strain at the end of transformation A→M (ε_{mf}) | 8.0% | |
| Strain at the start of transformation A→M (σₘₛ) | 500 | MPa |
| Stress at the end of transformation M→A (σ_{af}) | 250 | MPa |
| Transformation module (H) | 2500 | MPa |

The bilinear material law representing the superelastic behavior of nitinol is used to compute the response of a tridimensional nitinol structure. An hysteresis in the material law, whereby the path followed during unloading is not the same as that followed during loading, is observed. This property of the material will transpose itself to the behavior of the nitinol stent. In fact, it is observed during the numerical simulations that the response in contraction of the metallic armature does not follow the same path at the time of its deployment.

During the deployment of the stent by the inflatable balloon, the polymer's behavior may be characterized as being “elasto-plastic”. Until a certain stress value, referred to as the yield stress σγ is reached, the material behaves elastically without manifesting a plastic deformation. The initial rigidity of the material is given by the Young's modulus (E). When the stress exceeds the yield value, plastic strains are induced in the material and a residual deformation is thus obtained if the stress is subsequently taken back to zero. This plastic regime may be observed until rupture, that is when the deformation reaches a value ε_{B} referred to as the ultimate strain. The rigidity at the time of plastification is largely inferior to that obtained during the elastic behavior.

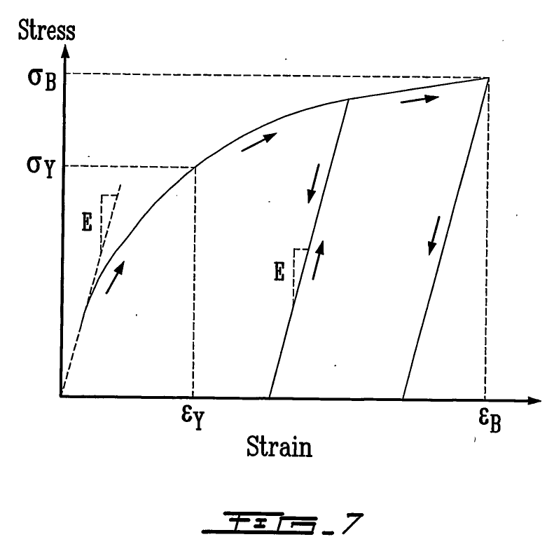

FIG. 7 schematizes a stress-strain relationship used in the calculations to model the polymeric matrix's behavior. The values of the different parameters used for the calculations are given in Table 4, as taken from the technical data for Bayers Makrolon Rx 2530.

TABLE 4

| | | |
|---|---|---|
| Elasticity modulus (E) | 2400 | MPa |
| Poisson coefficient (υ) | 0.3 | |
| Yield stress (S_{Y}) | 65 | MPa |
| Ultimate stress (S_{B}) | 75 | MPa |
| Yield strain (ε_{Y}) | 6% | |
| Ultimate strain (ε_{B}) | 120% | |

To calculate the behavior of the nitinol metallic armature, a finite element method may be employed, considering only half of a zigzag is considered, since during a uniform contraction, the nine zigzags form a cylindrical segment that is subjected to the same efforts and deformations. Therefore, it is not necessary to repeat the same calculations many times. A finite element mesh used for the analysis is constituted of 893 nodes and 2,955 elements in a tetrahedral form (pyramid with a triangular base). All these elements follow the law of nitinol's bilinear behavior described hereinbefore. To cause the deformation, punctual forces F are applied at the extremities of the semi-zigzag. Therefore, an increase in these forces yields a contraction of the stent. To take into account the fact that the real geometry is not only constituted by one semi-zigzag, but by a number of them, geometrical restrictions are introduced as “boundary conditions”. In the present case, these boundary conditions reflect the fact that the symmetry surfaces always lie on the same plane during the structure deformation.

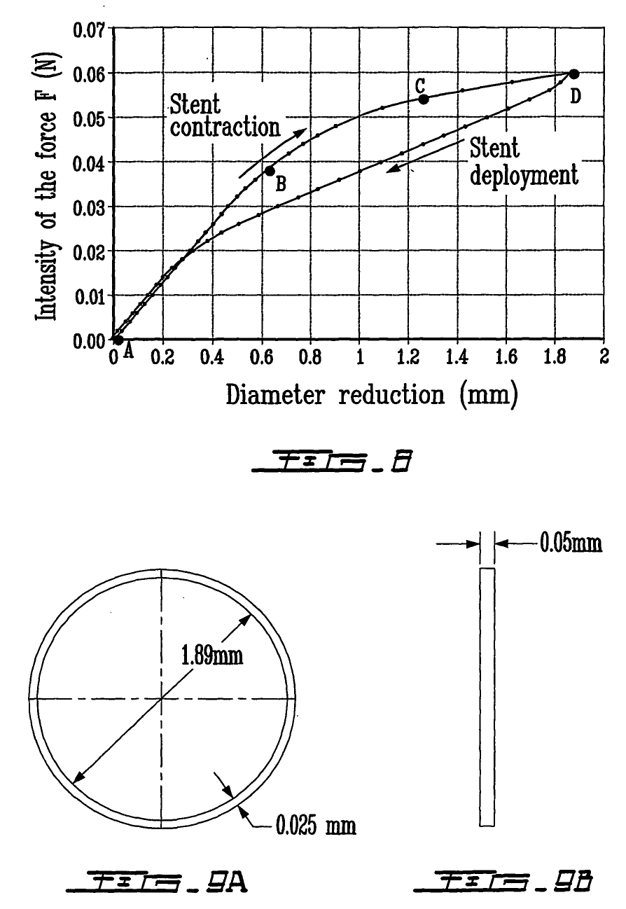

By increasing the intensity of the forces, the stent contracts itself and the decrease in diameter of the stent in relation to the applied force may then be calculated. The graph in FIG. 8 shows the evolution of the external diameter of the armature. The diameter has a value of 3.75 mm when the stent is in the completely deployed position. An hysteresis related to the fact that the stent follows a different path during the contraction and during the progressive deployment is clearly noticeable. The hysteresis is intrinsically considered in the bilinear material law, which simulates the super-elastic behavior of nitinol.

In addition to calculating the stent contraction in relation to the applied force, the finite element analysis allows estimating the stresses in the material. A stress rise near the extremities of the semi-zigzag is thus observed, while a central part thereof appears to be practically unsolicited. Maximal stresses generated in the structure are approximately 600 MPa, which corresponds to less than 5% of strain. Because nitinol is able to accommodate close to 8% of strain, it may then be concluded that this stent may be contracted to levels equivalent to those reached during the analysis without getting damaged and even further.

Returning to FIG. 6 of the appended drawings, clearly the addition of a polymeric matrix on the armature may be done in several ways adaptable by a person of ordinary skill. For example, as shown in FIG. 6A, the polymer may completely cover the metallic armature with a layer thereof. However, this solution appears technically inconvenient, since the metallic armature and the polymeric matrix would have more or less the same geometry, while the nitinol is approximately 75 times more rigid than Makrolon (rigidity module of 80 000 MPa for nitinol, compared to 1100 MPa for Makrolon). Therefore, it results that the layer of polymer would not be able to maintain the metallic armature in a contracted position.

Therefore, since the geometry is similar, the rigidity of the materials is roughly the same. Moreover, addition of this layer of polymer is intended to be performed when the stent is completely contracted. From the point of view of fabrication, mechanical means used to maintain the metallic armature in a contracted position may prevent the application of a uniform layer of polymer.

As FIG. 6B shows, a possible solution to obtain two structures of similar rigidity, the structures being the polymeric matrix and the metallic armature, comprises using rings of polymer. Since the polymer is clearly less rigid than nitinol, structures with a similar rigidity may be obtained by fortifying the polymeric matrix into a rigid geometry. Therefore, the nitinol armature is materially rigid and structurally flexible, while the polymeric ring is materially flexible and structurally rigid.

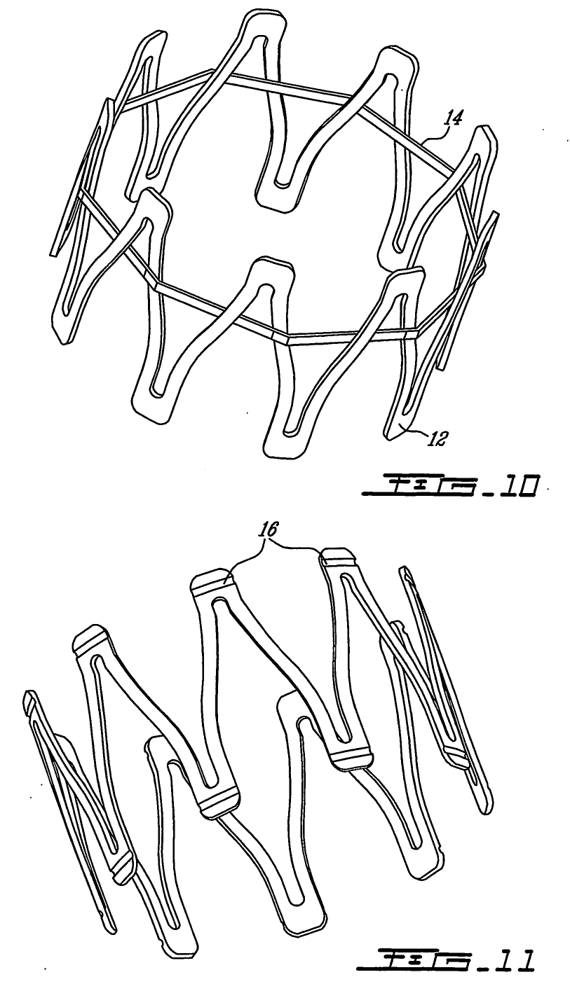

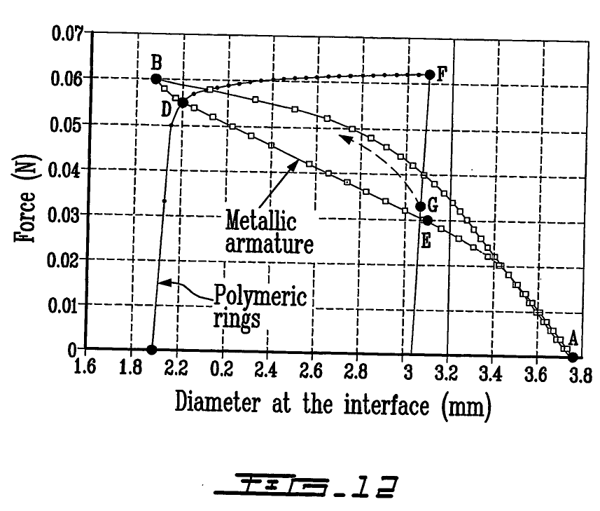

It is to be noted that the rings of polymer may be positioned in a number of alternative ways. For example, a complete coating 14 covering completely the armature 12 may be used as illustrated in FIG. 6 A, or the rings 14 may be braided around the armature 12 (see FIG. 10), or secured in slots 16 provided on the armature 12 (see FIG. 11).

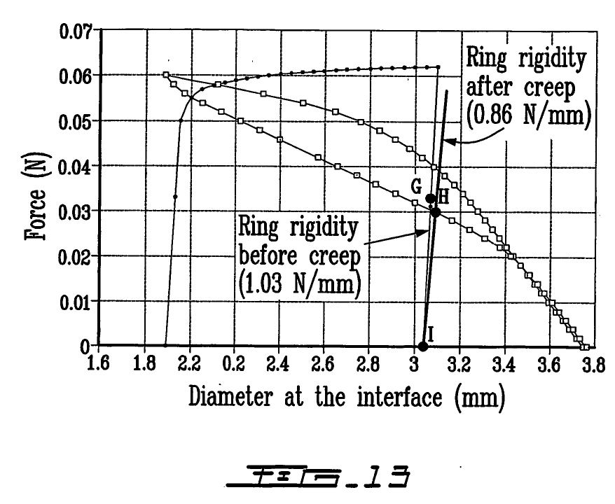

Dimensions of the polymeric ring-like structure yielding a desired global rigidity may be calculated. The present analysis is based on the interior diameter of the ring (1.89 mm), which corresponds to the exterior diameter of the metallic armature for the maximal contraction reached during the previous analysis. The other dimensions, being the thickness of the wall at 0.025 mm and the width of the ring at 0.05 mm, are determined by applying a calculation algorithm (FIG. 12). Finally, the material of the ring is exemplified here with Makrolon, the properties of which have been discussed hereinbefore. Of course, a person of ordinary skill will understand that the present invention may use alternative material in the polymer matrix.

For the calculation of the behavior of the ring, it may be assumed that the stress in a section is uniform. In fact, because of the geometry of the ring, its rigidity in flexion, referred to as radial crushing, is negligible in comparison to its rigidity in traction caused by the uniform dilation, i.e. increase in circumference. Therefore, the ring has a tendency to take the form of a polygon during its dilation by the inflatable balloon. When assuming that the ring dilates into a polygon form, the stress in the section of the ring may thus be considered simply as axial traction. Moreover, it is assumed that the ring is deformed by the reaction forces of the metallic armature on the ring, which are thus the equivalent of the forces F considered hereinabove to deform the nitinol armature.

The study of the behavior of the ring consists in evaluating the force F required to increase the diameter of the ring from an initial value φ₀ to a given value φ. Mathematical relations applicable to a regular polygon enable us to link the length of a side of the polygon S to its radius R

When the ring is deformed, the length S of the side of the polygon increases, and this from S₀. The imposed stress to the ring may then be calculated by the definition of engineering strain, that is, the ratio of the lengthening on the initial length:
ε=ΔL/L₀=(S−S₀)/S₀

The last notion that must be presented before presenting the calculation algorithm concerns the equilibrium of the forces. The force F must be balanced by the internal stresses a generated in the polymeric ring. However, it was discussed before that these stresses are supposed to be constant and normal at the section of the ring. Therefore, it is possible to write the following equilibrium equation where b and t are respectively the thickness and the width of the polymeric ring:
F=2σb t sin(20°)

By using the results presented previously, the following method may be used to estimate the behavior of a polymeric ring. The goal is to develop a method enabling to link the applied force F on the ring in relation to the diameter φ of the ring.

1) Impose a value to the diameter of the deformed ring φ

2) Calculate the length of the side of the polygon: S=0.342 φ

3) Calculate the stress in the ring by using the initial length S₀=0.646 mm:
ε=(S−S₀)/S₀
4) For a given strain value ε, deduct the stress σ with the help of the material law for Makrolon;
5) Calculate the force necessary to generate this stress with the help of the equilibrium equation F=2 σb t sin (20°) when b=0.05 mm and t=0.025 mm:
F=8.55·10^{4 σ}

In summary, starting with a given value for the interior diameter φ of the polymeric ring, it is possible to estimate the necessary force F to reach this diameter. Table 5 gives the results of the calculations that were made with the help of the algorithm using the 5 steps presented above. The diameter of the ring is dilated from the initial internal diameter of 1.89 mm to a diameter of 3.10 mm and then, the force F is pulled back to 0.

TABLE 5

| Φ (mm) | S (mm) | ε | σ (MPa) | F (N) |
|---|---|---|---|---|
| 1.89 | 0.646 | 0.000 | 0.0 | 0.000 |
| 1.92 | 0.657 | 0.016 | 38.5 | 0.033 |
| 1.95 | 0.667 | 0.032 | 58.5 | 0.050 |
| 2.00 | 0.684 | 0.059 | 64.9 | 0.055 |
| 2.05 | 0.701 | 0.085 | 66.7 | 0.057 |
| 2.10 | 0.718 | 0.112 | 67.6 | 0.058 |
| 2.15 | 0.735 | 0.138 | 68.3 | 0.058 |
| 2.20 | 0.752 | 0.165 | 68.9 | 0.059 |
| 2.25 | 0.770 | 0.191 | 69.4 | 0.059 |
| 2.30 | 0.787 | 0.218 | 70.0 | 0.060 |
| 2.35 | 0.804 | 0.244 | 70.3 | 0.060 |
| 2.40 | 0.821 | 0.271 | 70.6 | 0.060 |
| 2.45 | 0.838 | 0.297 | 70.9 | 0.061 |
| 2.50 | 0.855 | 0.324 | 71.2 | 0.061 |
| 2.55 | 0.872 | 0.350 | 71.4 | 0.061 |
| 2.60 | 0.889 | 0.376 | 71.6 | 0.061 |
| 2.65 | 0.906 | 0.403 | 71.7 | 0.061 |
| 2.70 | 0.923 | 0.429 | 71.9 | 0.061 |
| 2.75 | 0.940 | 0.456 | 72.0 | 0.062 |
| 2.80 | 0.958 | 0.482 | 72.2 | 0.062 |
| 2.85 | 0.975 | 0.509 | 72.3 | 0.062 |
| 2.90 | 0.992 | 0.535 | 72.5 | 0.062 |
| 2.95 | 1.009 | 0.562 | 72.6 | 0.062 |
| 3.00 | 1.026 | 0.588 | 72.7 | 0.062 |
| 3.05 | 1.043 | 0.615 | 72.9 | 0.062 |
| 3.10 | 1.060 | 0.641 | 73.0 | 0.062 |
| 3.07 | 1.050 | 0.626 | 36.5 | 0.031 |
| 3.04 | 1.041 | 0.611 | 0.0 | 0.000 |

Table 5 stresses the maximal strain reached during the analysis, that is 64.1% of strain. However, this value is largely inferior to the ultimate strain of 120% from which the fissuring or the rupture of the Makrolon may arise. The chosen conception is thus safe in relation to that aspect.

The non-linear behavior may be observed by considering the force applied on the ring in relation to the diameter φ thereof. It appears that the ring acts more or less like an elastic when the dilation force is inferior to 0.05 N. Beyond this value, plastification occurs and the ring deforms itself considerably under the effect of a very light increase in the applied force. Moreover, an elastic return (elastic springback) occurs when the force on the ring is completely released after having been dilated to a diameter of 3.10 mm. A residual deformation is also observed, because the ring does not return to its initial state with a diameter of 1.89 mm, but rather to a dilated state with a diameter of 3.04 mm. This irreversible behavior is the consequence of the elasto-plastic characteristic of the polymer.

The analysis carried-out previously may now be combined to determine the simultaneous behavior of the nitinol armature and the polymeric rings. A working model comprises two rings per zigzag segment of the metallic armature. If, for example, a metallic armature comprises 7 zigzag segments, 14 such rings are assumed on the metallic armature, the internal diameter of the rings being the same as the external diameter of the armature, and referred to as the interface diameter. Of course, it will be realized that depending on the polymer used, the shape and design of the ring-like members as well as the number of such ring-like members etc. may be varied.

FIG. 12 allows understanding the functioning of the stent comprising the nitinol armature and the polymeric rings. In this graphic, the nitinol armature is firstly completely deployed, as represented by point A in the graphic, and the diameter of the armature is 3.75 mm. The armature is then contracted to a diameter of 1.89 mm (point B), under a force F of 0.06 N. At that time, the non-deformed polymeric rings, which also have an internal diameter of 1.89 mm, are introduced around the metallic armature. The non-deformed rings correspond to point C on the graph. Then, the contraction force applied on the metallic armature is completely released, thereby reestablishing the equilibrium between the armature and the rings, which implies that the rings pass from points C to D, while the armature passes from points B to D. Point D represents the equilibrium state before the deployment commanded by an inflated balloon. The diameter of the structure is 2.0 mm. As observable in FIG. 12, such a design of the polymeric rings allows retaining the nitinol metallic armature in a contracted position.

The deployment of the stent in the artery is performed using an inflatable balloon. The interface diameter is then increased from the equilibrium position (2.0 mm) to a value of 3.1 mm. Thus the metallic armature passes from point D to point E, while the rings do the same from point D to point F. The balloon is then deflated and removed, which results in the armature and the rings reaching a second equilibrium position. A light contraction due to the elastic springback of the polymeric rings that pass from points F to G is observed, while the armature passes from points E to G according to a path describing an hysteresis (curved arrow, starting at point E). Point G represents the equilibrium position after the inflatable balloon has been deflated and removed from the artery. The interface diameter of the stent is then 3.07 mm. Clearly, in this example, the rings appear to sustain a great irreversible deformations during the inflation of the balloon.

At point G, the stent comprising the nitinol armature reinforced by the Makrolon rings is installed in the artery. The equilibrium forces are then approximately 0.032 N. According to computations, these forces generate stresses of about 37 MPa in the polymeric rings. According to the Makrolon 3100 creep data enumerated above, a loss in rigidity of the material of about 33% is observed when the material is subjected to stresses of some thousands psi (which represents about ten or so MPa) at a temperature neighboring that of the human body (104° F.) for a time lapse of 1000 hours (42 days). At point G, the rigidity of the ring is 1.03 N/mm, as determined from the slope of the curve. A rigidity of 0.68 N/mm may thus be set forward after 1000 hours at 37° C. FIG. 13 shows the variation in rigidity of the ring caused by the creep phenomenon. The loss of rigidity may be visualized graphically by the rotation of the slope in relation to the pivot 1. The new equilibrium position then becomes point H, which represents the intersection of the new rigidity of the ring with the curve modeling the increase of the diameter of the armature. Point H indicates a diameter of 3.09 mm after 1000 hours of creep, which represents an increase of 0.02 mm in relation to the diameter before creep.

The increase in diameter due to creep as exemplified hereinabove is rather weak. However, analyses demonstrate that it is possible to increase the diameter of a stent on a long period of time following the surgical intervention. The material exemplified here does not creep sufficiently to permit a desired increase of the diameter of the stent over a prolonged period of time following implantation. A loss of more than 90% of the rigidity of the polymeric material may allow a post-operation deployment of about 0.2 or 0.3 mm, which would come closer to the sought-out performances. Therefore, the use of a polymeric material having similar mechanical properties as the Makrolon exemplified herein, while being able to creep more, would enable the reaching of the objectives of the most preferred conceptions. For example, polyethylene such as DMDA-8920 polyethylene, which may have a loss of more than 90% of the rigidity, may allow a post-operation deployment of the stent.

The results mentioned above demonstrate the feasibility of a stent comprising a nitinol armature and polymeric bands. This stent is able to deploy itself on a long period of time (from several weeks to several months) in an independent and progressive fashion, once the surgical operation is completed. Moreover, the installation of the stent in the artery is performed using an inflatable balloon, which allows a control thereof.

Dimensioning of the different components of the stent as determined is then suggested. It is shown that the polymeric rings are important structural elements of the stent since they delay the deployment of the nitinol armature. It is shown for example that rings of 0.025 mm of thickness and 0.050 mm in width may assure a post-operation deployment of a Radius™ nitinol stent of 3.75 mm. According to the dimensions mentioned, these rings have the necessary rigidity to retain the metallic armature in a contracted position before the deployment by the inflatable balloon, while simultaneously still offering great irreversible deformations during the inflation of the balloon. The efficiency of the creep in allowing a retarded and progressive deployment may be tested experimentally in animals.

From the foregoing, it should now be apparent that the present invention is a cardiovascular balloon-deployable stent having a controlled radial expansion over time enabling its precise installation.

The stent of the present invention minimizes the damage to the vessel, which may be associated with an abrupt mechanical expansion.

Moreover, the stent of the present invention minimizes the springback elastic effect in view of its continuous pressure on the artery walls for a prolonged period of time, after the withdrawal of the balloon that has served for its initial deployment.

Other numerical analyses may further be realized to take into account the behavior of the artery during the deployment of the stent.

In addition to characterizing the creep properties of the polymeric material used, the analyses in animals may help to verify experimentally the global behavior of the stent. Prototypes may be made and tested.

As used herein, the term “cardiovascular” encompasses the coronary and peripheral vascular systems. Hence, the stents of the present invention are not limited to a use in coronary angioplasty. Indeed, the stents of the present invention find use in any disease or condition in which a stenting of a vessel or lumen would be beneficial.

Although the present invention has been described hereinabove by way of possible embodiments thereof, it may be modified without departing from the nature and teachings thereof as defined in the appended claims.

What is claimed is:

1. A balloon-deployable and controlled radially expandable stent comprising:
an armature comprising a first material having an elasticity allowing an expansion over time of said armature;
a matrix comprising a second material having a rigidity and a conformation allowing a retention of said armature in a contracted position;
said stent being deployed with the help of a balloon introduced into said armature, said balloon allowing an irreversible deformation of said matrix during inflation of said balloon and allowing expansion of the armature.

2. The stent of claim 1, wherein said armature and said matrix are structures of similar rigidity.

3. The stent of anyone of claims 1 and 2, wherein said first material is a shape memory alloy.

4. The stent of claim 3, wherein said metal shape memory alloy is nitinol.

5. The stent of claim 1, wherein said second material is a polymer and said deformation is plastic.

6. The stent according to anyone of claims 1 to 5, wherein said matrix is fortified into a rigid geometry by rings.

7. The sent according to claim 6, wherein said rings are selected in the group comprising a coating made of rings covering completely said armature, rings braided around said armature, and rings secured in slots provided on said armature.

8. The stent of anyone of claims 1 to 7, wherein said second material has a rigidity of at least 1000 MPa, a yield strain below about 8%, and an ultimate strain over about 100%.

9. The stent of anyone of claims 1 to 8, wherein said second material is selected in the group comprising a polycarbonate and a polyethylene.

10. The stent of anyone of claims 8 to 9, wherein said second material further exhibits creep properties allowing a minimum loss of 50% of an initial rigidity within 1000 hours.

11. The stent of anyone of claims 1 to 10, wherein the matrix conformation is annular.

12. The stent of anyone of claims 1 to 11, further comprising a retention sheath covering said matrix and said armature, and recuperating expansion forces of said armature by preventing a creep of said matrix.

13. A method of angioplasty in an artery of a patient comprising:
introducing and positioning in a vessel of the patient a self-deploying stent having a progressive deployment comprising an armature comprising a material having an elasticity allowing self-deployment of the armature; and a matrix comprising a second material having a rigidity and a conformation allowing a retention of the armature in a contracted position;
deploying the armature using a balloon delivered in the armature, the balloon ensuring an irreversible deformation of the matrix during inflation of the balloon and allowing a self-deployment of the armature; and
removing the balloon from the vessel;
whereby a progressive self-deployment of the armature allows a positioning of the armature at a predetermined position and a diminution of a risk of restenosis.

14. The method of claim 13, wherein the armature comprises a shape memory alloy.

15. The method of claim 14, wherein the shape memory alloy is nitinol.

16. The method of anyone of claims 13 to 15, wherein the second material is a polymer and wherein the deformation is plastic.

17. The method of claim 16, wherein the polymer has a rigidity of at least 1000 MPa, a yield strain below about 8%, and an ultimate strain over about 100%.

18. The method of anyone of claims 15 and 16, wherein the polymer is selected in the group comprising a polycarbonate and a polyethylene polymer.

19. The method of anyone of claims 17 and 18, wherein the polymer further exhibits creep properties characterized by a loss of rigidity of at least 50% of an initial rigidity thereof within 1000 hours.

20. The method of anyone of claims 13 to 19, further comprising before step a) an expulsion of said stent from a retention sheath covering the matrix and the armature and recuperating the expansion forces of the armature by preventing a creep of the matrix.

21. The stent of claim 1, wherein said first material has radio-opacity and rigidity properties comparable to metal.
